(19) United States
(12) Patent Application Publication
(10) Pub. No.: US 2004/0210270 A1
(43) Pub. Date: Oct. 21, 2004

(54) High frequency pulse generator for an implantable neurostimulator

(76) Inventor: John Erickson, Plano, TX (US)

Correspondence Address: Koestner Bertani LLP, P.O. Box 26780, Austin, TX, 75755, US

(21) Appl. No.: 10/627,231

(22) Filed: Jul. 25, 2003

Related U.S. Application Data
(60) Provisional application No. 60/398,740, filed on Jul. 26, 2002.
(60) Provisional application No. 60/398,704, filed on Jul. 26, 2002.
(60) Provisional application No. 60/398,749, filed on Jul. 26, 2002.
(60) Provisional application No. 60/400,366, filed on Aug. 1, 2002.

(51) Int. Cl. A61N 1/18

(52) U.S. Cl. 607/46

(57) ABSTRACT

The invention is directed to a method and apparatus for electrically stimulating tissue. Many typical devices suffer from charge build-up on blocking capacitors when stimulating tissue at higher frequencies. The invention actively drives the discharge of the blocking capacitors, reducing the discharge time. As a result, stimulating pulses may be delivered at higher frequencies. Actively discharging the blocking capacitors may be accomplished with an asymmetric reverse pulse. This reverse pulse may be provided by a switching circuitry that reverses the polarity applied to the electrodes or couples a pulse from a second pulse generator to the electrodes.

### RELATED APPLICATIONS

This application claims the benefit of U.S. Provisional Patent Application No. 60/398,740 entitled, “High Frequency Pulse Generator for an Implantable Neurostimulator” filed Jul. 26, 2002. Additionally, this application incorporates by reference the prior U.S. provisional application Nos. 60/398,704 entitled, “Method and System for Energy Conservation in Implantable Stimulation Devices” filed Jul. 26, 2002; 60/398,749 entitled, “Method and Apparatus for Providing Complex Tissue Stimulation Patterns” filed Jul. 26, 2002; and 60/400,366 entitled, “Bendable Needle with Removable Stylet” filed Aug. 1, 2002.

### TECHNICAL FIELD OF THE INVENTION

This invention relates in general to pulsed electrical tissue stimulation. More specifically, this invention relates to actively discharging blocking capacitors to enable the use of increased frequencies in tissue stimulation.

### BACKGROUND OF THE INVENTION

Electronic stimulation systems may be used to control pain or motor disorders. Such systems have also been used to stimulate bone growth. For example, spinal cord stimulation (SCS) is one technique that has been used for pain management. Typical SCS systems feature a pulse generator coupled to one or more leads having a plurality of electrodes. The leads may be positioned within a patient's epidural space, parallel to the axis of the spinal cord. The leads' electrodes are used to deliver an electric field to a specific region of the spinal cord or surrounding tissue. Applying the electric field across one or more nerve bundles and/or nerve roots can produce paresthesia, or a subjective sensation of numbness, tingling or “pins and needles,” at the affected nerves' dermatomes. This paresthesia, if properly directed and produced at the necessary levels, can “mask” certain forms of chronic pain.

The focus, characteristics and intensity of the generated electric field are determined by the electrode configuration (i.e., the polarity, if any, assumed by each electrode) and the electric pulse waveform (collectively “stimulation setting”). The waveform properties include, at least, a stimulation frequency, a stimulation pulse width and phase information.

SCS systems are of two types. The most common system is a totally implanted pulse generator (IPG). An IPG consists of a surgically implanted, internally-powered pulse generator and, typically, a single multi-electrode lead. The internalized power source limits the life of these systems to between two and four years. After the power source is expended, the patient is required to undergo replacement surgery to continue electrical stimulation.

The second type of SCS system is a radio frequency (RF) system. An RF system consists of a surgically implanted, passive receiver and a transmitter which is worn externally. The transmitter is connected to an antenna which is positioned externally, over the site of the implanted receiver. In operation, the transmitter communicates through an RF signal, to the implanted receiver. Just as with the IPG system, electrical stimulation is delivered via implanted leads. However, RF systems typically possess greater power resources, thereby enabling RF systems to utilize multiple leads.

In addition to the use of this technology for pain management, some researchers believe that SCS may have beneficial application in obtaining relief from and/or controlling the physical effects of peripheral vascular disease (PVD), angina pectoris, and various motor disorders.

However, these typical electronic stimulation devices are limited in their ability to stimulate using high frequencies. Typical systems have a set of blocking capacitors between the pulse generating source and the leads. These blocking capacitors build charge during a pulse and discharge in the reverse direction after the pulse. The discharging prevents corrosion of the leads and provides a zero net current flow through the tissue. The discharge occurs at lower voltages and lower currents, preventing stimulation of the tissue during discharge. However, the discharge period is considerably longer than the pulse. Thus, the discharge period limits the frequency of the pulse.

At higher frequency, the capacitors do not fully discharge and instead build charge. As the capacitors build charge, the stimulation pulse experienced by the tissue is changed. The pulse may lose current density, voltage, current, and/or power, depending on the configuration of the stimulator. As a result, the stimulation pulses may ineffectually or cease to stimulate the tissue.

Similar problems arises when multiple leads or stimulation settings are used. Typically, electrodes used in one setting are fully discharged before stimulation using another setting. If the discharge period is long or charge builds for higher frequency pulses, the rate of change between settings may be limited.

As such, many typical stimulation systems suffer from deficiencies in providing high frequency stimulation. Many other problems and disadvantages of the prior art will become apparent to one skilled in the art after comparing such prior art with the present invention as described herein.

### SUMMARY OF THE INVENTION

Aspects of the invention may be found in an apparatus for actively discharging blocking capacitors of a tissue stimulator. The invention may be used to actively discharge the capacitors of a neurostimulation or neuromodulation device. The tissue stimulation or modulation device may have one or more pulse generators, switching circuitry, one or more electrodes, and one or more blocking capacitors. The pulse generators may be coupled to the blocking capacitors which may be coupled to the electrodes. The device and/or the electrodes may be placed in vivo to provide a stimulation pulse to the tissue. The stimulation device may be configurable to provide the stimulation pulse of a given amplitude, charge, and pulse width. Further, the stimulation device may be re-configurable to provide a reverse pulse of opposite charge to the blocking capacitors and/or same electrodes. This reverse pulse of opposite charge may have a longer pulse width and or a lower amplitude than the stimulation pulse. As such, the stimulation pulse and the reverse pulse are asymmetric. In this manner, the reverse pulse may act to accelerate the discharge of the blocking capacitors thereby enabling the use of higher frequency pulses. As such, the device may deliver stimulation pulses at rates of 2-5000 Hz. However, the rates may be higher or lower than this range.

Additional aspects of the invention may be found in a switching circuitry for configuring and reconfiguring the stimulation device. The switching circuitry may accomplish a stimulation pulse and reverse pulse by internally switching the node to which the blocking capacitors and/or electrodes are coupled. Alternately, the nodes to the switching circuitry may be switched. In another exemplary embodiment, a second pulse generator may be used to generate the reverse pulse.

A further aspect of the invention may be found in a charge integrator connected to the switching circuitry or included in the stimulation device. The charge integrator may determine the total charge delivered by a pulse. In this manner, the charge delivered by a stimulation pulse may be matched with that of a reverse pulse.

Further aspects of the invention may be found in a method for actively discharging the blocking capacitors of a stimulation device. After a stimulation pulse, a reverse pulse may be used to actively drive the discharge of the blocking capacitors. The reverse pulse may have an opposite charge, lower amplitude, and/or longer pulse width than the stimulation pulse. The reverse pulse and stimulation pulse may deliver the same total or integral charge to the circuitry.

As such, an apparatus and method for actively discharging blocking capacitors is described. Other aspects, advantages and novel features of the present invention will become apparent from the detailed description of the invention when considered in conjunction with the accompanying drawings.

### BRIEF DESCRIPTION OF DRAWINGS

For a more complete understanding of the present invention and advantages thereof, reference is now made to the following description taken in conjunction with the accompanying drawings in which like reference numbers indicate like features and wherein:

FIG. 1 is a schematic diagram depicting a stimulation device;

FIG. 2 is a pictorial depicting exemplary embodiment of leads and electrodes;

FIG. 3A is a diagram depicting the placement of leads and electrodes in vivo;

FIG. 3B is a diagram depicting the placement of leads and electrodes in vivo;

FIG. 3C is a diagram depicting the placement of leads and electrodes in vivo;

FIG. 4A is a diagram depicting regions affected by spinal stimulation;

FIG. 4B is a diagram depicting regions affected by spinal stimulation;

FIG. 4C is a diagram depicting regions affected by spinal stimulation;

FIG. 5 is a schematic block diagram depicting an exemplary embodiment of a stimulation device, according to the invention;

FIG. 6 is a schematic block diagram depicting an exemplary embodiment of a stimulation device, as seen in FIG. 5;

FIG. 7A is a pictorial depicting current flow in tissue;

FIG. 7B is a pictorial depicting current flow in tissue;

FIG. 7C is a pictorial depicting current flow in tissue;

FIG. 7D is a pictorial depicting current flow in tissue;

FIG. 7E is a pictorial depicting current flow in tissue;

FIG. 8 is a graph depicting efficacious pulse stimulations;

FIG. 9 is a schematic diagram depicting an exemplary embodiment of the stimulation device as seen in FIG. 5;

FIG. 10 is a graph depicting an exemplary pulse response;

FIG. 11A is a graph depicting a pulse response;

FIG. 11B is a graph depicting a pulse response;

FIG. 11C is a graph depicting a pulse response;

FIG. 12 is a graph of an applied pulse;

FIG. 13A is a schematic diagram of an exemplary embodiment of the stimulation device as seen in FIG. 5;

FIG. 13B is a schematic diagram of an exemplary embodiment of the stimulation device as seen in FIG. 5;

FIG. 14 is a schematic diagram of an exemplary embodiment of the stimulation device as seen in FIG. 5;

FIG. 15 is a schematic diagram of an exemplary embodiment of the stimulation device as seen in FIG. 5;

FIG. 16 is a schematic diagram of an exemplary embodiment of the stimulation device as seen in FIG. 5; and

FIG. 17 is a block flow diagram of an exemplary method for use by the system as seen in FIG. 5.

Corresponding reference numerals indicate corresponding parts throughout the several views of the drawings.

### DETAILED DESCRIPTION OF THE INVENTION

Several conditions may benefit from electrical pulse stimulation or modulation of tissue. These conditions may include pain, bone growth, cardiac arrest and arrhythmias, peripheral vascular disease (PVD), angina pectoris, and various motor disorders. The electrical pulse stimulation may be delivered by a lead with several electrodes placed near the tissue to be stimulated. The lead may be connected to a stimulation device which is either implanted corporally or external to the body.

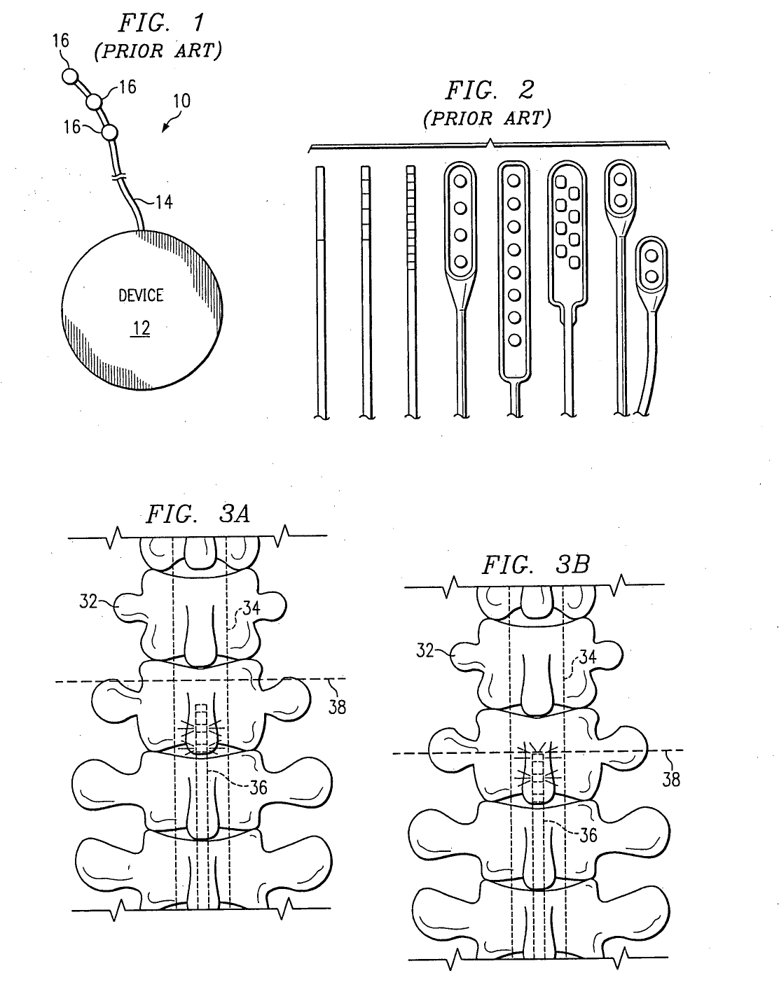

FIG. 1 is an exemplary embodiment of a stimulation system 10. The system may include the stimulation device 12 connected to a lead 14. The lead 14 may terminate in one or more electrodes 16. For example, the device 12 may be implanted in a patient. The lead 14 may extend from the device 12 and terminate near the stimulated tissue. In this manner, the electrodes 16 may be place near the tissue to be stimulated.

In one exemplary embodiment, the device 12 may be a neuromodulator or neurostimulation device. The leads 14 may, for example, extend into the spinal foremen and the epidural space about the spinal cord. As a result, the electrodes may be positioned to stimulate nervous tissue about the spinal cord or extending from the spinal cord. In this position, the electrodes may be activated to induce paresthesia or manage motor conditions.

FIG. 2 depicts various electrodes as may be found on leads. The electrodes may be cylindrical or flat. Further, the electrodes may be addressed and activated individually. Selectively applying a voltage across or a current between various electrodes may provide a differing effect on the tissue and in the perception of the patient. For example, one configuration of activated electrodes may serve to create paresthesis in one location while activation of another set may induce paresthesia in a perceptively different location. Selection of a set of electrodes, their charge configuration, and the pulse characteristic may be included in a stimulation set.

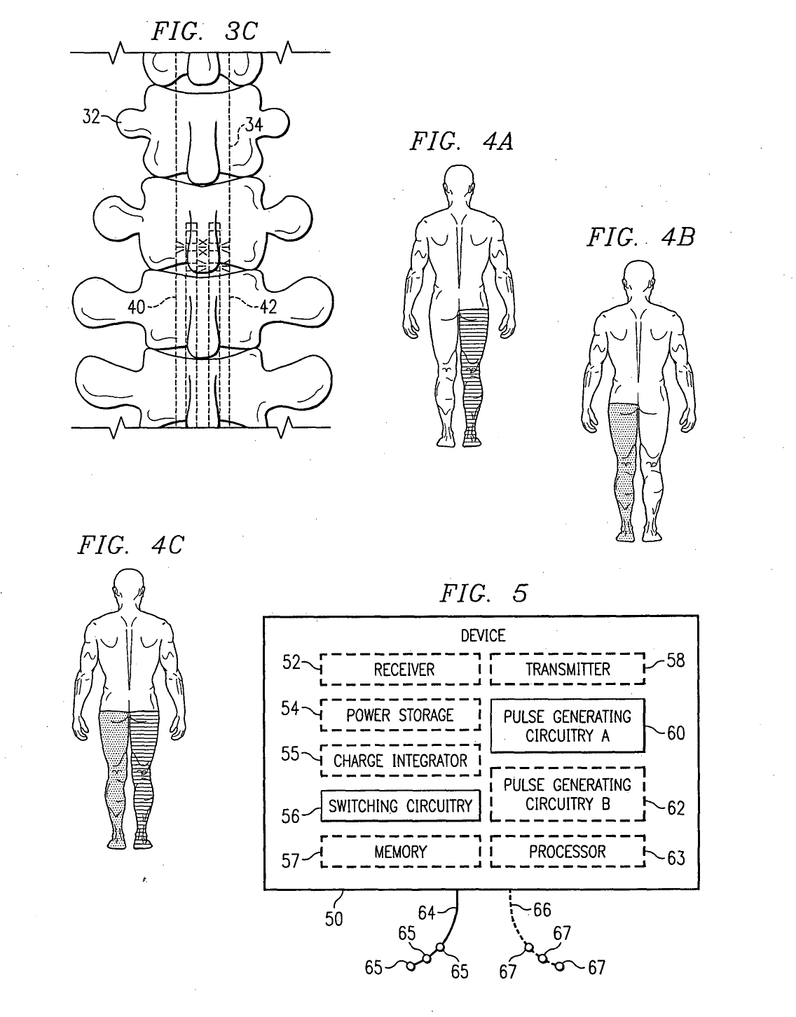

In one exemplary embodiment, a lead may be inserted in the spinal foramen and terminate at a desired location. FIG. 3A depicts a lead 36 terminating at the location 38 in the spinal foreman 34. By activating a pair of electrodes, the cord may be stimulated to produce a desired effect. However, the lead may shift over time or as a result of movement by the patient. In this case, the termination location 38 of the lead 36 and consequently the electrodes may change. As a result, a differing set of electrodes may be used to produce the same effect.

In another exemplary embodiment, more than one lead 40 and 42 may be placed in the spinal foramen 34 as shown in FIG. 3C. Electrodes may be activated in proximal leads 40 and 42 to achieve desired result. However, one or more leads of varying type and electrode configuration may be placed in various corporal locations to achieve various results.

In one exemplary embodiment, a desired result may be the relief of leg pain or the control of motor conditions. Placement of electrodes about the spinal column may produce a response in nervous tissue leading to the legs. With this placement, activation of one set of electrodes may affect the right leg as seen in FIG. 4A. However, activation of another set of electrodes may affect the left leg as seen in FIG. 4B. If the two sets of electrodes were to be alternately activated, conditions in both legs may be managed. However, alternating sets of electrodes may also be used to mitigate differing conditions in a single location, the same conditions in the same location as the patient moves, or other combinations of conditions, locations, given varying body postures of the patient.

During a stimulation cycle, a blocking capacitor leading to the electrodes must be discharged prior to switching stimulation sets. If blocking capacitors associated with a first set are not discharged, they may discharge in conjunction with the next set. If this were to occur, the electrical flow field would be altered and the response of the tissue may change.

Further, the blocking capacitors must be discharged prior to subsequent stimulation pulses on the same electrode set. If the blocking capacitors used during a first stimulation pulse are not discharged, the remaining electrical charge from the first pulse will interact with the next pulse, altering the actual pulse characteristics experienced by the electrodes. This may prevent induction of the desired effect.

In both cases, failure to fully discharge the blocking capacitors causes the net flow of charge through the tissue to be non zero. As a result, the electrodes may corrode.

FIG. 5 depicts a stimulation device 50 enabled to actively discharge the blocking capacitors. The stimulation device 50 may have a receiver 52, transmitter 58, power storage 54, charge integrator 55, switching circuitry 56, memory 57, pulse generators 60 and 62, and processor 63, among others. Further, the device 50 may be coupled to one or more leads 64 and 66. These leads may terminate in one or more electrodes 65 and 67. However, some, all, or none of the components may be included in the device 50. Further, these components may be together, separate, or in various combinations, among others.

The receiver 52 may take various forms. These forms may include a circuitry, antenna, or coil, among others. The receiver 52 may or may not function to receive instructions and data. Further, the receiver 52 may or may not function to receive power that may be used by the device 50 and/or stored in the power storage 54. Similarly, the transmitter 58 may take various forms including a circuitry, antenna, or coil, among others. The transmitter 58 may function to transmit data and/or instructions. However, the receiver 52 and transmitter 58 may or may not be included or may be together, separate, or combine various components, among others.

The power storage 54 may take various forms. These forms may include various batteries.

The switching circuitry 56 may take various forms. These forms may include various contacts, relays, and switching matrices, among others. Further, the switching circuitry 56 may or may not include one or more blocking capacitors associated with connections to the leads. These blocking capacitors may block direct connection to the leads and/or function to build charge that may be discharged between stimulation pulses. In addition, the switching circuitry 56 may or may not be integrated with the charge integrator 55.

The memory 57 may take various forms. These forms may include various forms of random access memory, read-only memory, and flash memory, among others. The memory 57 may be accessible by the switching circuitry 56, and/or the processor 63.

The processor 63 may take various forms. These forms may include logic circuitry or microprocessors, among others. The processor 63 may function to monitor, deliver, and control delivery of the modulation or stimulation signal. Further, the processor 63 may manipulate the switching circuitry 56 and determine the amount of charge delivered with the charge integrator 55.

The one or more pulse generators 60 and 62 may take various forms. These forms may include a clock driven circuitry or an oscillating circuitry, among others. The pulse generator(s) 60 and 62 may deliver a electric or electromagnetic signal through the switching circuitry 56 to the leads 64 and 66 and electrodes 65 and 67. The signal may be modulated by circuitry associated with the switching circuitry 56, and/or the processor 63 to manipulate characteristics of the signal including amplitude, frequency, polarity, and pulse width, among others.

The leads 64 and 66 and the electrodes 65 and 67 may take various forms. These forms may include those shown above, among others.

In one exemplary embodiment, the device 50, the leads 64 and 66, and the electrodes 65 and 67 are implanted in the body of a patient. The leads 64 and 66 extend from the device into the spinal foramen, terminating about the spinal cord. Stimulation sets of electrodes 65 and 67 may then be selected and activated. During activation, the switching circuitry applies an electrical pulse to blocking capacitors and the select set of electrodes 65 and 67. In this manner, a pulse or flow field of electricity is applied to stimulate the nervous tissue. Following each activation or pulse, a reverse pulse is applied to the blocking capacitors and the select set of electrodes 65 and 67. The reverse pulse may be asymmetric relative to the stimulation pulse and may be selected to prevent further stimulation of the tissue. For example, the reverse pulse may have a longer pulse width and/or lower amplitude. Similarly, the total charge delivered by the stimulation pulse may equal that of the reverse pulse. With the reverse pulse, charge may be driven from the blocking capacitors faster without further stimulating the nervous tissue.

However, various embodiments and uses of the device 50, the leads 64 and 66, and the electrodes 65 and 67 may be envisaged. Further, various embodiments of the device 50 and its elements may be envisaged.

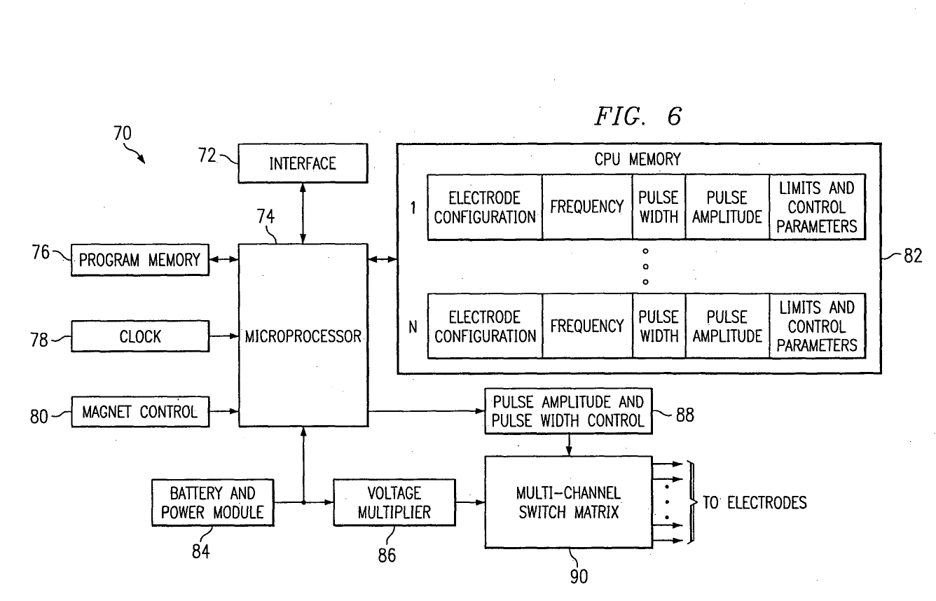

FIG. 6 is a schematic block diagram depicting another exemplary embodiment of the system. This exemplary embodiment 70 may have a microprocessor 74, an interface 72, a program memory 76, a clock 78, a magnet control 80, a power module 84, a voltage multiplier 86, a pulse amplitude and width control 88, a CPU memory 82, and a multi-channel switch matrix 90. However, these components may or may not be included and may be together, separate, or in various combinations.

The microprocessor 74 may take the form of various processors and logic circuitry. The microprocessor 74 may function to control pulse stimulations in accordance with settings 1 through N stored in the CPU memory 82. Further, the microprocessor 74 may function in accordance with programs stored in the program memory 76. The microprocessor 74 may also function to determine the total amount of charge delivered by a given pulse.

The program memory 76 may take various forms. These forms may include RAM, ROM, flash memory, and other storage mediums among others. Further, the program memory 76 may be programmed using interfaces 72.

These interfaces 72 may be accessed prior to implanting to program the microprocessor 74, program memory 76, and or CPU memory 82. These forms may include ports or connections to handheld circuitry, computers, keyboards, displays, and program storage, among others. Alternately, the interfaces 72 may include means for interaction and programming after implanting.

A clock 78 may be coupled to the microprocessor 74. The clock may provide a signal by which the microprocessor operates and/or uses in creating stimulation pulses.

A magnet control 80 may also interface with the microprocessor. The magnet control 80 may function to start or stop stimulation pulses. Alternately, a receiver or other means may be used. This receiver may or may not function to provide programming instruction, power charge, and on/off signals.

The system 70 may also have a power supply or battery 84. This power supply 80 may function to power the various circuitries such as the clock 78, microprocessor 74, program memory 76, and CPU memory 82, among others. Further, the power supply 80 may be used in generating the stimulation pulses. As such, the power supply may be coupled to the microprocessor 74, a voltage multiplier, and/or a switch matrix 90.

The CPU memory 82 may consist of RAM, ROM, Flash or other storage medium. The CPU memory 82 may store stimulation settings 1 through N. These stimulation settings may include electrode configuration, pulse frequency, pulse width, pulse amplitude, and other limits and control parameters. The reverse pulse parameters may or may not be stored in the CPU memory 82 and may or may not be associated with each of the stimulation settings 1 through N. The microprocessor 74 uses these stimulation settings and parameters in configuring the switch matrix 90 and producing stimulation pulses and reverse pulses.

The switch matrix 90 may permit more than one lead with more than one electrode to be connected to the system 70. The switch matrix 90 functions with other components to selectively stimulate varying sets of electrodes with various pulse characteristics. Further, the switch matrix may include one or more blocking capacitors coupled between the power source and the leads.

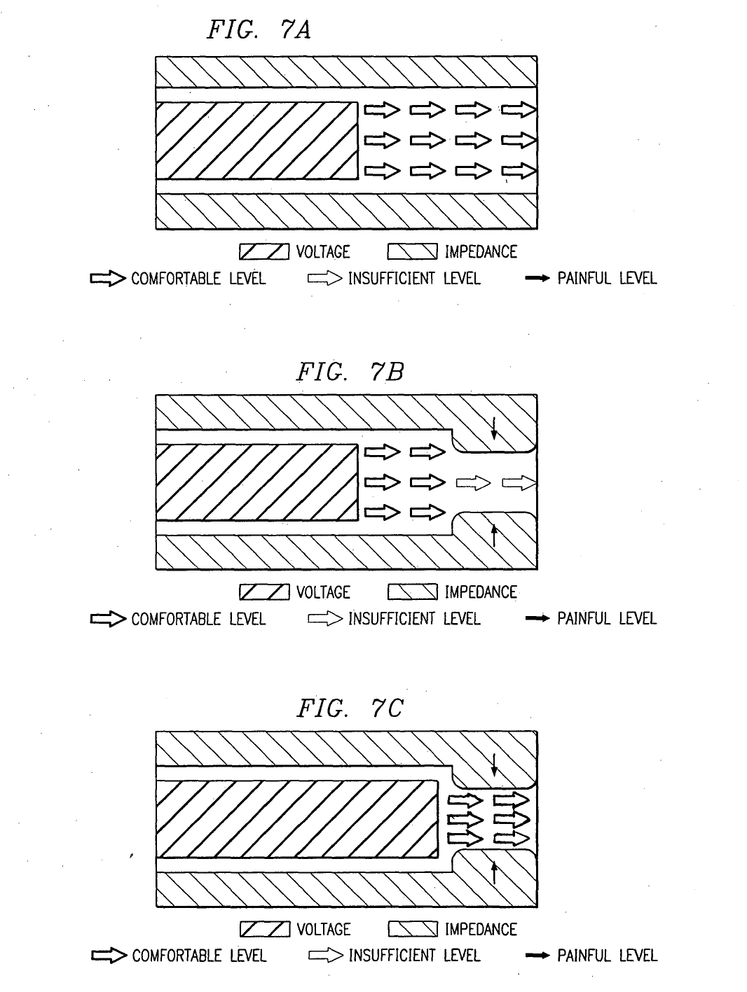

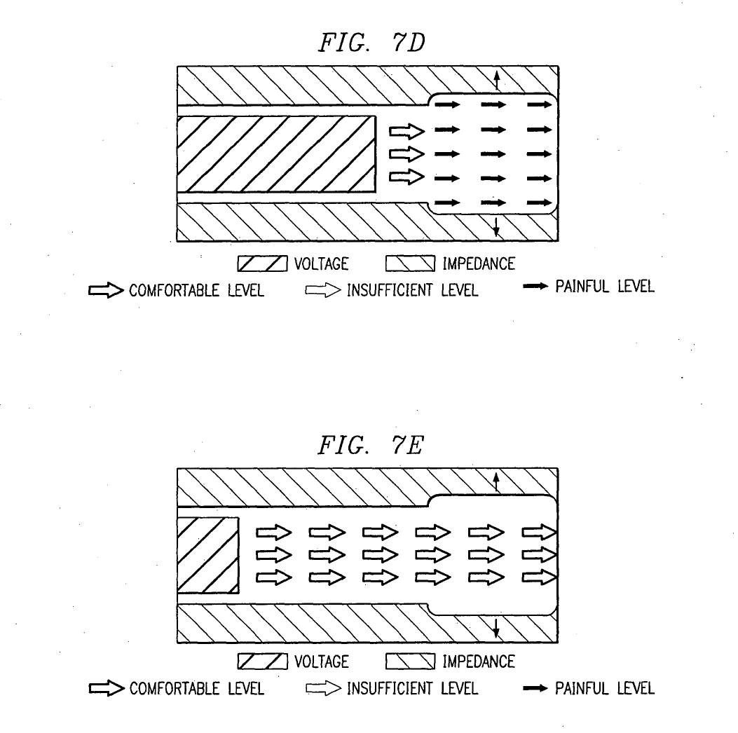

The stimulation device may operate in various manners to provide a stimulation pulse to the tissue. One manner is to provide a constant voltage pulse and another is a constant current pulse. FIG. 7A depicts pictorially, the current, voltage, impedance relationship. In tissue, a voltage may be applied across a set of electrodes. The circuit including the tissue provides resistance and impedance. This impedances may vary between tissue types and with tissue scarring or growth. In these pictorials, the current is metaphorically depicted as a flow field in a tube. For a constant voltage mechanism an increase in impedance causes a restriction and less current flows through the metaphoric tube as seen in FIG. 7B. The reduced current may not properly stimulate the tissue. With a constant current configuration, the current remains constant and the voltage increases as seen in FIG. 7C.

FIGS. 7D and 7E depict the two configurations for a decrease in impedance. A decrease in impedance reduces the metaphoric restriction. For the constant voltage configuration excess current may flow through the tissue as seen in FIG. 70. This excess current may be uncomfortable to the patient. However, as seen in FIG. 7E, the voltage may be reduced to keep the current constant.

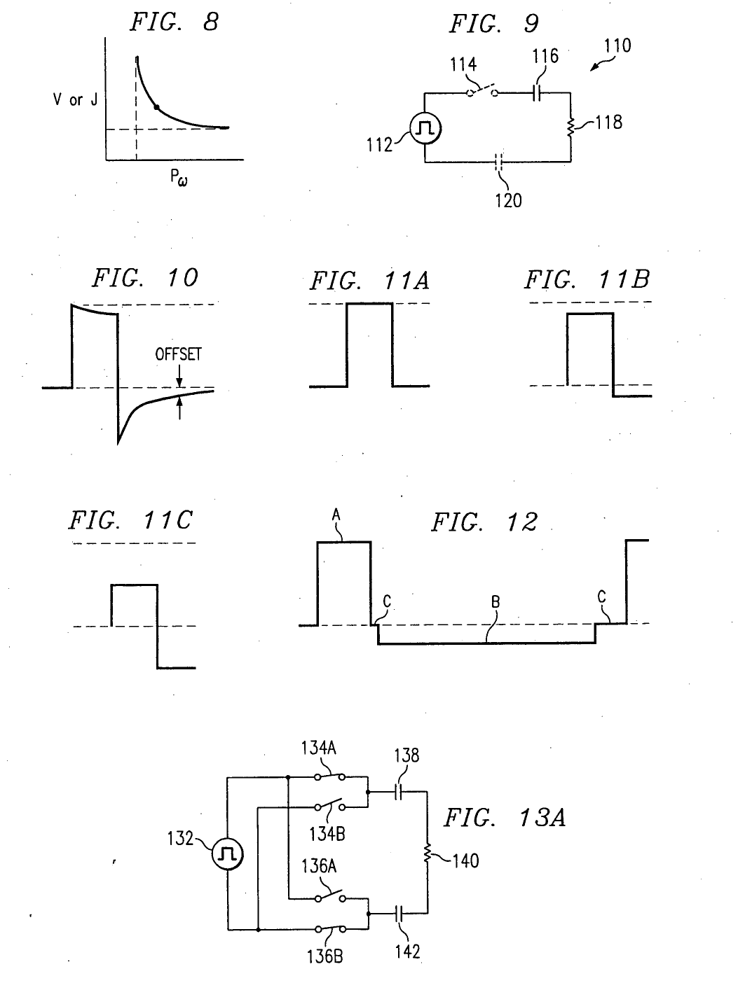

FIG. 8 depicts the relationship between the pulse width and the voltage or current density. To achieve stimulation, the pulse width may be wider for lower voltages and currents. Conversely, the pulse width may be smaller for higher voltages and currents. However, an optimum may exist as depicted by a point. In some neural modulation applications, the optimal stimulation occurs in the range of 3-6 volts and 200-500 □s pulse width. However, various tissues may be stimulated using varying voltages and pulse widths.

Further, the frequency between pulses may influence the effect of the stimulation, as well. Desired frequencies of pulse stimulation may range between 2 and 5000 Hz. High frequency pulses may include frequencies about 250 Hz depending on the various capacitances and resistances of the circuits. However, the frequencies may be more or less than these ranges.

As described above, in one embodiment, the pulse is delivered through electrodes located about the tissue to be stimulated. FIG. 9 depicts a conceptual circuit of the stimulation. The circuit 110 has a pulse generator 112, an optional switch 114, one or more blocking capacitors 116 and 120, a resistance 118. The resistance 118 represents the cumulative resistance and may include circuitry, connections, leads, electrodes, and tissue, among others.

When switch 114 is closed, the pulse generator 112 generates a pulse. The pulse may, for example, have a constant voltage or a constant current. During the pulse, charge accumulates or builds on the capacitors 116 and 120. Once the generator completes the pulse, capacitors 116 and 120 discharge the electrical energy through the tissue as represented by resistor 118. This discharge prevents corrosion on the electrodes and provides for a zero net current flow through the tissue. The period for this discharge is a function of the capacitance, accumulated charge and resistance.

FIG. 10 depicts the flow response as seen by the tissue for a constant voltage pulse. The pulse causes a charge to build on the blocking capacitors. Upon completion of the pulse, the charge on the blocking capacitors discharges through the tissue. However, if a second pulse occurs prior to complete discharge, an off-set charge remains on the blocking capacitors.

FIGS. 11B, and 11C depict the effective pulse of successive pulses with incomplete discharge as seen at the electrode. An ideal pulse, as seen in FIG. 11A effectively returns to a base state. As charge builds on the blocking capacitors, subsequent pulses effectively provide lower amplitudes at the electrode. FIG. 11B shows a subsequent pulse with effectively a slight offset due to the residual charge on the capacitor. FIG. 11C shows a subsequent pulse at 50% of its desired value.

If an inverse pulse is applied to the to circuit 110 in FIG. 9, discharge of the capacitors occurs quickly, preventing charge buildup. An exemplary pulse set may be seen in FIG. 12. This pulse set may be a constant voltage pulse set, constant current pulse set, or take various shapes and pulse configurations including ramping, sinusoidal, saw tooth, or various combinations, among others.

In FIG. 12, a stimulation pulse A is applied to the circuit followed by a reverse pulse B. Interspaced between stimulation pulses and reverse pulses may be a quiescent period C. The reverse pulse B may have a smaller amplitude than the stimulation pulse A. Further, the reverse pulse B may have a longer pulse width than the stimulation pulse A. Moreover, the reverse pulse and the stimulation pulse may have the same integrated current flow or together, produce a zero net current flow through the tissue and electrodes. The zero net flow may prevent electrode corrosion.

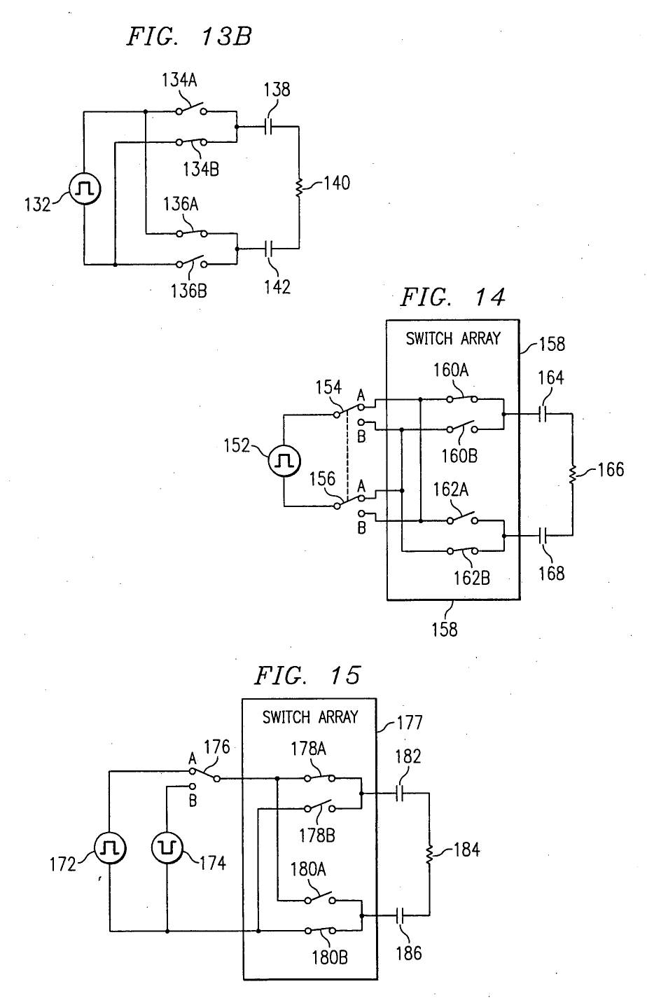

FIGS. 13A and 13B show an embodiment of a circuit for producing the stimulating pulse and the reverse pulse. A generator 132 produces an electrical stimulating pulse. This stimulation pulse may be a constant voltage or constant current pulse. As seen in FIG. 13A, the pulse is directed through closed switches 134A and 136B, blocking capacitors 138 and 142, and a resistance 140 representing the combined resistance of the tissue and the circuitry. As explained above, the pulse causes a charge to accumulate on the blocking capacitors 138 and 142.

After the stimulation pulse, the switches are reconfigured to effectively reverse the charge applied to the blocking capacitors 138 and 142. Then, the pulse generator 132 generates a second pulse. As seen in FIG. 13B, the second pulse travels through closed switches 134B and 136A, the capacitors 138 and 142, and the resistance 140. By closing switches 134B and 136A and opening the switches 134A and 136B, the charge is effectively reversed, driving the charge from the capacitors through the resistance 140. With the driving force of the reverse pulse, the blocking capacitors 138 and 142 discharge more rapidly than they would independent of the reverse pulse. The rate of discharge and thus the period required for complete discharge may be manipulated by changing the characteristics of the reverse pulse. These characteristics include the pulse width and amplitude.

In an exemplary constant current embodiment, the reverse pulse would have an amplitude of half the stimulation pulse. In an alternate example, the reverse pulse may have an amplitude of a quarter of the stimulation pulse. However, the reverse pulse may have an amplitude more or less than these examples.

Further, the pulse width of the reverse pulse may be twice or four times that of the stimulation pulse. However, the pulse width may have a length more or less that these examples. Moreover, the product of the pulse width and amplitudes of the two pulses may be the same. In this manner, the net flow of charge will be zero. For example, the reverse pulse may have an amplitude of half the stimulation pulse and a pulse width twice that of the stimulation pulse. Alternately, the reverse pulse may have an amplitude of a quarter of that of the stimulation pulse and a pulse width twice that of the stimulation pulse.

Greater reverse pulse widths reduce the frequency by which pulses may be generated. However, the long pulses widths permit low amplitude reverse pulses. Lower amplitude reverse pulses are less likely to stimulate the tissue. In this manner, the circuitry may be driven to discharge the blocking capacitors while preventing unwanted secondary stimulation of the tissue.

Alternately, a constant voltage pulse or a variety of pulse shapes controlled with voltage, current or a combination may be used. The total current flow through the tissue caused by the stimulation pulse may be matched by the reverse pulse. In this manner, stimulation pulses may be spaced close together and corrosion of the electrodes may be prevented. However, various pulse embodiments may be envisaged.

In another exemplary embodiment, the switching array may be held in one configuration and the charge reversed on the inputs to the switching array. FIG. 14 depicts a circuitry with a pulse generator 152, switches 154 and 156 leading to the switching array, a switching array 158 with switches 160A, 160B, 162A, and 162B, blocking capacitors 164 and 168, and a resistance 166. Again, the resistance may include tissue and various circuitry.

As seen, the switching array may be configured to selectively couple a set of electrodes and blocking capacitors 164 and 168 to node A of switches 154 and 156. The pulse generator then generates a stimulation pulse that passes through the blocking capacitors 164 and 168 and the resistance 166. After the stimulation pulse, the switches 154 and 156 may be switched to connect to node B of switched 154 and 156, thus effectively reversing the charge placed on the circuitry. A subsequent reverse pulse may be directed through the switching array 158, effectively driving the discharge of the blocking capacitors 164 and 168.

This method is especially useful for larger switching arrays with varying numbers and configurations of electrodes. A first configuration can be established by the switching array 158. The stimulation pulse may be delivered. Then, the charge can be reversed and the blocking capacitors 164 and 168 actively discharged. Subsequently, the array 158 may be reconfigured to select a differing set of electrodes. However, the blocking capacitors on the first set must be discharged prior to stimulation using the second set or the stimulation field will be altered by the discharging of the blocking capacitors associated with the first set or by charge remaining on blocking capacitors associated with electrodes common to both sets.

In a further exemplary embodiment, a second pulse generator may be used to generate the reverse pulse. FIG. 15 depicts a circuitry with two pulse generators 172 and 174. Similar to the circuit seen in FIG. 14, the switching array 177 may be configured to a first set of electrodes. This first set is exemplified by the closed switches 178A and 180B and the open switches 178B and 180A.

The pulse generator 172 may generate a stimulation pulse. The pulse may travel through switch 176 connected to node A, the switching array 177, the blocking capacitors 182 and 186, and the resistance 184. Subsequently, a reverse pulse may be generated by the pulse generator 174. The switch 176 may be connected to node B, permitting a pulse of opposite charge to drive the discharge of the blocking capacitors 182 and 186 through the configured switching array 177.

Further, after the discharge, the switch 176 may be connected to node A for a subsequent pulse. Additionally, the switching array 177 may be configured to permit stimulation and activation of a differing set of blocking capacitors and electrodes.

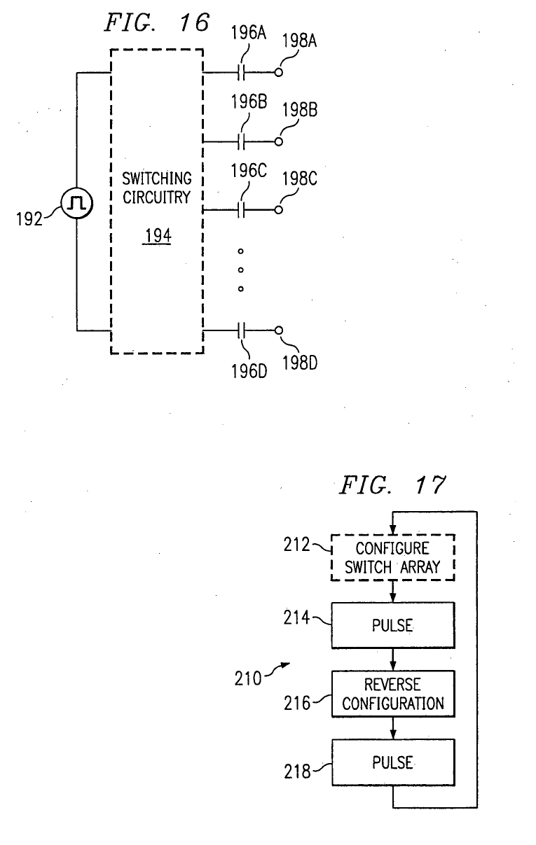

FIG. 16 shows another exemplary circuitry. The circuitry may have one or more generators 192, a switching circuitry 194, and a set of blocking capacitors 196 and electrodes 198. The electrodes may be placed in proximity to a desired tissue. Subsequently, the switching circuitry 194 may be configured to selectively couple subsets of blocking capacitors 196 and electrodes 198 to the one or more generators 192. In the manners described in relation to FIGS. 13A, 13B, 14, and 15, among others, the switching circuitry 194 and the generators may be configured to deliver stimulation pulses and subsequently reverse pulses to the selected subset of blocking capacitors 196 and electrodes 198.

For example, the switching circuitry 194 may be configured to selectively apply a positive charge to blocking capacitor 196A and electrode 198A. Further, the switching circuitry 194 may be configured to selectively apply a negative charge to blocking capacitor 196B and electrode 198B. The pulse generator 192 may then stimulate the tissue in proximity to these electrodes. A subsequent reverse pulse may be directed to these blocking capacitors and electrodes by either reversing the charge or by generating a pulse of opposite charge.

A second stimulation pulse may then be delivered or a second subset of blocking capacitors and electrodes may be selected. However, various combinations of varying numbers of electrodes may be selected.

Further, the switching circuitry may take various forms. The blocking capacitors may be included together or separately from the switching circuitry. Further, the blocking capacitors may or may not be uniquely connected with dedicated electrodes. Moreover, various embodiments and implementations of the pulse generator, switching circuitry, blocking capacitors, and electrodes may be envisaged.

Turning to FIG. 17, a method 210 is shown for enabling higher frequency pulse delivery. A switching array may be configured as seen in a block 212. Various means of switching and configuring an array may be envisaged. These means may include hardware, software, and combinations of hardware and software. However, the switching array may or may not require configuration.

Subsequently, a pulse may be generated as seen in a block 214. This pulse may be the stimulation pulse. The switching array may then be reconfigured to enable a reverse pulse. Alternately, a second generator may be coupled to the switching array to generate a reverse pulse.

The system may then provide a reverse pulse to drive the discharge of the blocking capacitors as seen in a block 218. The reverse pulse may have a lower amplitude than the stimulation pulse. Further, the reverse pulse may have a longer pulse width than the stimulation pulse. Together, the reverse pulse and stimulation pulse may deliver a zero net current to the circuitry. The integrated total of the current flow for the reverse pulse may equal that of the stimulation pulse.

Subsequently, the switching array may be reconfigured to deliver a second pulse to set of electrodes. Alternately, the switching array may be configured to deliver a pulse to a second set of electrodes.

In this manner, the circuitry may deliver pulses to a given subset of electrodes at a higher frequency without buildup of charge on the blocking capacitors. Further, the circuitry may switch between stimulation settings with a higher frequency without buildup of charge on the capacitors or alteration of the expected charge flow field.

As such, a stimulation device is described. In view of the above detailed description of the present invention and associated drawings, other modifications and variations will now become apparent to those skilled in the art. It should also be apparent that such other modifications and variations may be effected without departing from the spirit and scope of the present invention as set forth in the claims which follow.

1. A method for stimulating living tissue(s), comprising the steps of:
producing a first stimulator pulse with a first pulse generator;
delivering the first stimulator pulse to the living tissue(s) through electrodes electrically coupled to the stimulator with at least one lead, and wherein at least one blocking capacitor electrically coupled to the first pulse generator the provides a net zero current flow through the living tissues(s);
generating a reverse pulse that discharges the at least one blocking capacitor in order to shorten the at least one blocking capacitor's discharge period; and
producing a subsequent stimulator pulse with the pulse generator, wherein the subsequent stimulator pulse is delivered to the living tissue(s) at the end of the at least one capacitor's shortened discharge period.

2. The method of claim 1, wherein the reverse pulse is generated by a second pulse generator.

3. The method of claim 1, wherein the first stimulator pulse and the reverse pulse are assymetrical.

4. The method of claim 1, wherein an absolute total charge delivered by the first stimulation pulse equals an absolute total charge delivered by the reverse pulse.

5. The method of claim 1, wherein a switching network operates on the output of the first pulse generator to generate the reverse pulse.

6. The method of claim 5, wherein the switching networks reverse electrical connections to output nodes of the first pulse generator.

7. The method of claim 1, wherein stimulator pulses are applied to the living tissue(s) at a frequency greater than about 250 Hz without building charge on the at least one blocking capacitors.

8. The method of claim 1, wherein the discharge period is equal to or less than the stimulator pulses' pulse width.

9. The method of claim 1, wherein the steps are repeated with the subsequent stimulator pulse becoming the first stimulator pulse in order to produce high frequency stimulation pulses.

10. The method of claim 1, wherein the living tissue(s) comprise spinal cord tissues and wherein the stimulator pulses applied to the spinal cord tissues manage pain.

11. The method of claim 1, further comprising the step of implanting the first pulse generator, at least one lead, at least one blocking capacitor and electrodes within a living organism.

12. The method of claim 1, wherein the living tissue(s) comprise at least one nerve bundle.

13. A neurostimulator, comprising:
a first pulse generator that outputs a first stimulator pulse;
at least one blocking capacitor electrically coupled to the first pulse generator output, wherein the at least one blocking capacitor is electrically coupled to the first pulse generator in order to provide a net zero current flow through living tissues, and wherein a reverse pulse discharges the at least one blocking capacitor in order to shorten the at least one blocking capacitor's discharge period; and
at least one implanted lead electrically coupled to the output of the pulse generator that delivers the first stimulator pulse to electrodes proximate to living tissue to be stimulated, and wherein a subsequent stimulator pulse generated by the first pulse generator is delivered to the living tissue when the at least one blocking capacitor's discharge period is complete.

14. The neurostimulator of claim 13, wherein the reverse pulse is generated by a second pulse generator.

15. The neurostimulator of claim 13, further comprising a switching network that reverses electrical connections to output nodes of the first pulse generator to produce the reverse pulse.

16. The neurostimulator of claim 13, wherein the first stimulator pulse and the reverse pulse are assymetrical.

17. The neurostimulator of claim 13, wherein an absolute total charge delivered by the first stimulation pulse equals an absolute total charge delivered by the reverse pulse.

18. The neurostimulator of claim 13, wherein stimulator pulses are applied to the living tissue(s) at a frequency greater than about 250 Hz without building charge on the at least one blocking capacitors.

19. The neurostimulator of claim 13, wherein the discharge period is equal to or less than the stimulator pulses' pulse width.

20. The neurostimulator of claim 13, wherein the subsequent stimulator pulse becomes the first stimulator pulse in order to produce high frequency stimulation pulse patterns.

21. The neurostimulator of claim 13, wherein the living tissue comprises spinal cord tissues and wherein the stimulator pulses applied to the spinal cord tissues manage pain.

22. The neurostimulator of claim 13, wherein the first pulse generator, at least one lead, at least one blocking capacitor and electrodes are implantable within a living organism.

23. An implantable neurostimulator, comprising:
a first pulse generator that outputs a first stimulator pulse and a reverse stimulator pulse, wherein an absolute total charge delivered by the first stimulation pulse equals an absolute total charge delivered by the reverse pulse; and
at least one blocking capacitor electrically coupled to the first pulse generator outputs, wherein the at least one blocking capacitor provides a net zero current flow through living tissues, and wherein a reverse pulse is applied to and discharges the at least one blocking capacitor in order to shorten the at least one blocking capacitor's discharge period.

24. The implantable neurostimulator of claim 23, wherein the implantable stimulator electrically couples to at least one implanted lead in order to deliver the first stimulator pulse to electrodes proximate to living tissue, and wherein a subsequent stimulator pulse generated by the first pulse generator is delivered to the living tissue when the at least one blocking capacitor's discharge period is complete.

25. An implantable neurostimulator, comprising:
a first pulse generator that outputs a first stimulator pulse;
a second pulse generator that outputs a reverse stimulator pulse, wherein an absolute total charge delivered by the first stimulation pulse equals an absolute total charge delivered by the reverse pulse; and
at least one blocking capacitor electrically coupled to the first pulse generator's and second pulse generator's outputs, wherein the at least one blocking capacitor provides a net zero current flow through living tissues, and wherein a reverse pulse is applied to and discharges the at least one blocking capacitor in order to shorten the at least one blocking capacitor's discharge period.

26. The implantable neurostimulator of claim 25, wherein the implantable stimulator electrically couples to at least one implanted lead in order to deliver the first stimulator pulse to electrodes proximate to living tissue to be stimulated, and wherein a subsequent stimulator pulse generated by the first pulse generator is delivered to the living tissue when the at least one blocking capacitor's discharge period is complete.

27. The implantable neurostimulator of claim 25, wherein the first stimulator pulse and reverse stimulator pulse differ in pulse width and/or pulse anplitude.

28. An implantable neurostimulator that stimulate tissues with a high frequency stimulation pulse pattern, comprising:
a first pulse generator, having a first positive node and a first negative node;
a second pulse generator, having a second positive node and a second negative node;
an array of electrodes implanted proximate to one or more nerve bundles;
an array of capacitors;
a switching circuit coupled to the array of electrodes and the array of capacitors, wherein the switching circuit delivers a stimulation pulse to the one or more tissues, and wherein the stimulation pulse builds charge on the array of capacitors; and wherein the switching circuit actively discharges the array of capacitors with a reverse pulse.

29. The implanatable neurostimulator of claim 28, further comprising:
a charge integrator, the charge integrator determining the total amount of charge delivered to the one or more tissues for a given pulse.

30. The implanatable neurostimulator of claim 28, wherein the absolute value of a total charge delivered by the stimulation pulse equals the absolute value of a total charge delivered by the reverse pulse.

31. The implanatable neurostimulator of claim 28, wherein the stimulation pulse is a constant voltage pulse.

32. The implanatable neurostimulator of claim 28, wherein the stimulation pulse is a constant current pulse.

33. The implanatable neurostimulator of claim 28, wherein an amplitude of the reverse pulse has a lower absolute amplitude than an amplitude of the stimulation pulse.

34. The implanatable neurostimulator of claim 28, wherein a width of the reverse pulse is greater than a width of the stimulation.
